(19) United States
(12) Patent Application Publication
(10) Pub. No.: US 2022/0087739 A1
(43) Pub. Date: Mar. 24, 2022

(54) ENT INSTRUMENT WITH EXPANDABLE ABLATION FEATURE

(71) Applicants: Acclarent, Inc., Irvine, CA (US); Biosense Webster (Israel) Ltd., Yokneam (IL)

(72) Inventors: Jetmir Palushi, Irvine, CA (US); Henry F. Salazar, Pico Rivera, CA (US); Hany Abdelwahed, Irvine, CA (US); Assaf Govari, Haifa (IL); Vadim Gliner, Haifa (IL); Andres C. Altmann, Haifa (IL); Christopher T. Beeckler, Brea, CA (US); Shubhayu Basu, Anaheim, CA (US); Madison K. Vanosdoll, Irvine, CA (US); Marc Dean, Fort Worth, TX (US)

(21) Appl. No.: 17/404,088

(22) Filed: Aug. 17, 2021

Related U.S. Application Data
(60) Provisional application No. 63/080,066, filed on Sep. 18, 2020.

(51) Int. Cl. A61B 18/14; A61B 5/06; A61B 1/233; A61B 1/05

(52) U.S. Cl. CPC A61B 18/1492; A61B 5/062; A61B 2018/00327; A61B 1/05; A61B 2018/1465; A61B 1/233

(57) ABSTRACT

A surgical instrument includes a sheath and an ablation catheter disposed within the sheath. The sheath is configured to be inserted into a cavity of a patient's head. The ablation catheter extends along a longitudinal axis and includes at least one expandable ablation member configured to selectively radially expand outwardly from a non-expanded state to an expanded state for selectively ablating tissue within the patient's head. The ablation catheter is selectively translatable relative to the sheath between a proximal retracted position and a distal extended position. In the retracted position, the at least one expandable ablation member is housed within the sheath to thereby prevent the at least one expandable ablation member from radially expanding outwardly. In the extended position, the at least one expandable ablation member is exposed from the sheath to thereby permit the at least one expandable ablation member to radially expand outwardly for contacting tissue.

### PRIORITY

This application claims priority to U.S. Provisional Pat. App. No. 63/080,066, entitled “ENT Instrument with Expandable Ablation Feature,” filed Sep. 18, 2020, the disclosure of which is incorporated by reference herein, in its entirety.

### BACKGROUND

Rhinitis is a medical condition that presents as irritation and inflammation of the mucous membrane within the nasal cavity. The inflammation results in the generation of excessive amounts of mucus, which can cause runny nose, nasal congestion, sneezing, and/or post-nasal drip. Allergenic rhinitis is an allergic reaction to environmental factors such as airborne allergens, while non-allergenic (or “vasomotor”) rhinitis is a chronic condition that presents independently of environmental factors. Conventional treatments for rhinitis include antihistamines, topical or systemic corticosteroids, and topical anticholinergics, for example.

For cases of intractable rhinitis in which the symptoms are severe and persistent, an additional treatment option is the surgical removal of a portion of the vidian (or “pterygoid”) nerve—a procedure known as vidian neurectomy. The theoretical basis for vidian neurectomy is that rhinitis is caused by an imbalance between parasympathetic and sympathetic innervation of the nasal cavity, and the resultant over stimulation of mucous glands of the mucous membrane. Vidian neurectomy aims to disrupt this imbalance and reduce nasal mucus secretions via surgical treatment of the vidian nerve. However, in some instances, vidian neurectomy can cause collateral damage to the lacrimal gland, which is innervated by the vidian nerve. Such damage to the lacrimal gland may result in long-term health complications for the patient, such as chronic dry eye. Posterior nasal neurectomy, or surgical removal of a portion of the posterior nasal nerves, may be an effective alternative to vidian neurectomy for treating intractable rhinitis.

FIG. 1 depicts a left sagittal view of a portion of a patient's head, showing the nasal cavity (10), the frontal sinus (12), the sphenoid sinus (14), and the sphenoid bone (16). The nasal cavity (10) is bounded laterally by the nasal wall (18), which includes an inferior turbinate (20), a middle turbinate (22), and a superior turbinate (24). The vidian nerve (32) resides within the vidian (or “pterygoid”) canal (30), which is defined in part by the sphenoid bone (16) and is located posterior to the sphenoid sinus (14), approximately in alignment with the middle turbinate (22). The vidian nerve (32) is formed at its posterior end by the junction of the greater petrosal nerve (34) and the deep petrosal nerve (36); and joins at its anterior end with the pterygopalatine ganglion (38), which is responsible for regulating blood flow to the nasal mucosa. The posterior nasal nerves (40) join with the pterygopalatine ganglion (38) and extend through the region surrounding the inferior turbinate (20).

While instruments and methods for performing vidian neurectomies, posterior nasal neurectomies, and turbinate reductions are known, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a left sagittal view of a portion of a patient's head, showing details of certain paranasal sinuses and nerves, including the vidian nerve and the posterior nasal nerve;

FIG. 2 depicts a schematic view of an exemplary surgery navigation system being used on a patient seated in an exemplary medical procedure chair;

FIG. 3 depicts a schematic perspective view of an exemplary surgical system that includes an RF ablation instrument for use with the surgery navigation system of FIG. 2, showing an ablation catheter of the RF ablation instrument in a proximal retracted position relative to a sheath of the RF ablation instrument;

FIG. 4 depicts a disassembled perspective view of a distal portion of the RF ablation instrument of FIG. 3, showing an expandable ablation member of the ablation catheter in a non-expanded state;

FIG. 5A depicts a partial perspective view of a distal portion of the RF ablation instrument of FIG. 3, showing the ablation catheter in the proximal retracted position relative to the sheath with the expandable ablation member in the non-expanded state;

FIG. 5B depicts a partial perspective view of the distal portion of the RF ablation instrument of FIG. 3, showing the ablation catheter in a distal extended position relative to the sheath with the expandable ablation member in the non-expanded state;

FIG. 5C depicts a partial perspective view of the distal portion of the RF ablation instrument of FIG. 3, showing the ablation catheter in a distal extended position relative to the sheath with the expandable ablation member in an expanded state;

FIG. 6 depicts a partial perspective view of a distal portion of another exemplary RF ablation instrument suitable for use with the RF surgical system of FIG. 3, showing an ablation catheter of the RF ablation instrument in a distal extended position relative to a sheath of the RF ablation instrument with an expandable ablation member of the ablation catheter in an expanded state, and further showing the sheath including a pair of cameras for visualizing the expandable ablation member;

FIG. 7A depicts a partial perspective view of a distal portion of another exemplary RF ablation instrument suitable for use with the RF surgical system of FIG. 3, showing an ablation catheter of the RF ablation instrument in a distal extended position relative to a sheath of the RF ablation instrument with an expandable ablation member of the ablation catheter in an expanded state, and further showing an adjustable camera of the RF ablation instrument in a distal extended position relative to the expandable ablation member for visualizing a patient's anatomy distal of the ablation catheter;

FIG. 7B depicts a partial perspective view of the distal portion of the RF ablation instrument of FIG. 7A, showing the adjustable camera in a proximal retracted position relative to the expandable ablation member for visualizing the expandable ablation member;

FIG. 8A depicts a partial perspective view of a distal portion of another exemplary RF ablation instrument suitable for use with the RF surgical system of FIG. 3, showing an ablation catheter of the RF ablation instrument in a proximal retracted position relative to a sheath of the RF ablation instrument with expandable ablation members of the ablation catheter in respective non-expanded states;

FIG. 8B depicts a partial perspective view of the distal portion of the RF ablation instrument of FIG. 8A, showing the ablation catheter in a distal extended position relative to the sheath with the expanded ablation members in respective expanded states;

FIG. 9 depicts a partial perspective view of a distal portion of another exemplary RF ablation instrument suitable for use with the RF surgical system of FIG. 3, showing an ablation catheter of the RF ablation instrument in a distal extended position relative to a sheath of the RF ablation instrument with expandable ablation members of the ablation catheter in respective expanded states, and further showing one of the expandable ablation members positioned at a distal tip of the ablation catheter for contacting tissue distal of the distal tip;

FIG. 10A depicts a partial side elevational view of a distal portion of another exemplary RF ablation instrument suitable for use with the RF surgical system of FIG. 3, showing an ablation catheter of the RF ablation instrument in a distal extended position relative to a sheath of the RF ablation instrument with expandable ablation members of the ablation catheter in respective expanded states, the expandable ablation members being selectively translatable relative to each other along the shaft of the ablation catheter for adjusting an ablation region of tissue, and further showing the proximal expandable ablation member in a first proximal position and the distal expandable ablation member in a first distal position;

FIG. 10B depicts a partial side elevational view of the distal portion of the RF ablation instrument of FIG. 10A, showing the proximal expandable ablation member in a second proximal position and the distal expandable ablation member in the first distal position;

FIG. 10C depicts a partial side elevational view of the distal portion of the RF ablation instrument of FIG. 10A, showing the proximal expandable ablation member in the second proximal position and the distal expandable ablation member in a second distal position;

FIG. 11A depicts a left sagittal view of a portion of a patient's head, showing insertion of the RF ablation instrument of FIG. 3 into the patient's nasal cavity in the region of a posterior nasal nerve, and further showing the ablation catheter in the proximal retracted position relative to the sheath with the expandable ablation member in the non-expanded state; and

FIG. 11B depicts a left sagittal view of the portion of the patient's head with the RF ablation instrument of FIG. 3 inserted in the patient's nasal cavity in the region of the posterior nasal nerve, and further showing the ablation catheter in the distal extended position relative to the sheath with the expandable ablation member in the expanded state to deliver RF energy to a portion of the posterior nasal nerve to thereby ablate the nerve portion.

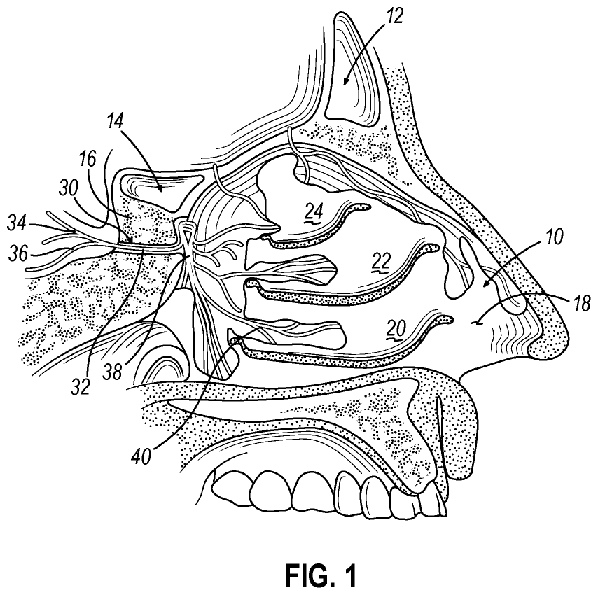

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

I. Exemplary Image Guided Surgery Navigation System

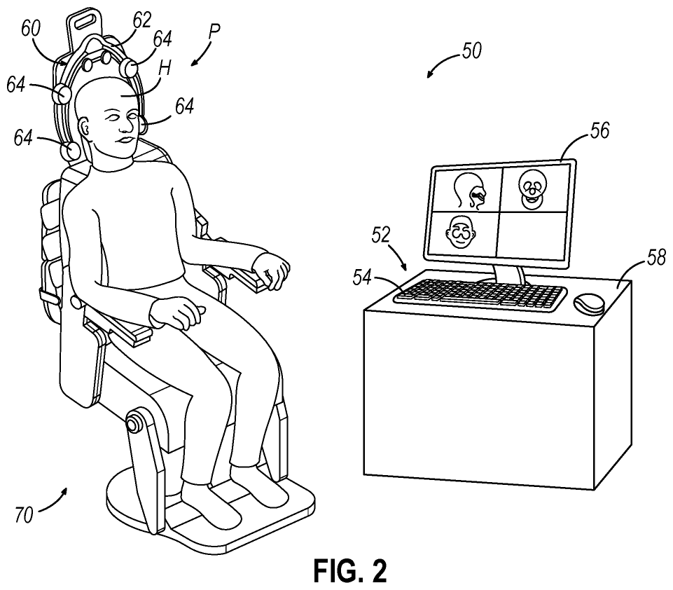

When performing a medical procedure within a head (H) of a patient (P), it may be desirable to have information regarding the position of an instrument within the head (H) of the patient (P), particularly when the instrument is in a location where it is difficult or impossible to obtain an endoscopic view of a working element of the instrument within the head (H) of the patient (P). FIG. 2 shows an exemplary IGS navigation system (50) enabling an ENT procedure to be performed using image guidance. In addition to or in lieu of having the components and operability described herein IGS navigation system (50) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; and U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein.

IGS navigation system (50) of the present example comprises a field generator assembly (60), which comprises set of magnetic field generators (64) that are integrated into a horseshoe-shaped frame (62). Field generators (64) are operable to generate alternating magnetic fields of different frequencies around the head (H) of the patient (P). An instrument, such as any of the RF ablation instruments described below, may be inserted into the head (H) of the patient (P). Such an instrument may be a standalone device or may be positioned on an end effector. In the present example, frame (62) is mounted to a chair (70), with the patient (P) being seated in the chair (70) such that frame (62) is located adjacent to the head (H) of the patient (P). By way of example only, chair (70) and/or field generator assembly (60) may be configured and operable in accordance with at least some of the teachings of U.S. Pat. No. 10,561,370, entitled “Apparatus to Secure Field Generating Device to Chair,” Issued Feb. 18, 2020, the disclosure of which is incorporated by reference herein.

IGS navigation system (50) of the present example further comprises a processor (52), which controls field generators (64) and other elements of IGS navigation system (50). For instance, processor (52) is operable to drive field generators (64) to generate alternating electromagnetic fields; and process signals from the instrument to determine the location of a navigation sensor in the instrument within the head (H) of the patient (P). Processor (52) comprises a processing unit (e.g., a set of electronic circuits arranged to evaluate and execute software instructions using combinational logic circuitry or other similar circuitry) communicating with one or more memories. Processor (52) of the present example is mounted in a console (58), which comprises operating controls (54) that include a keypad and/or a pointing device such as a mouse or trackball. A physician uses operating controls (54) to interact with processor (52) while performing the surgical procedure.

While not shown, the instrument may include a navigation sensor that is responsive to positioning within the alternating magnetic fields generated by field generators (64). A coupling unit (not shown) may be secured to the proximal end of the instrument and may be configured to provide communication of data and other signals between console (58) and the instrument. The coupling unit may provide wired or wireless communication of data and other signals.

In some versions, the navigation sensor of the instrument may comprise at least one coil at or near the distal end of the instrument. When such a coil is positioned within an alternating electromagnetic field generated by field generators (64), the alternating magnetic field may generate electrical current in the coil, and this electrical current may be communicated along the electrical conduit(s) in the instrument and further to processor (52) via the coupling unit. This phenomenon may enable IGS navigation system (50) to determine the location of the distal end of the instrument within a three-dimensional space (i.e., within the head (H) of the patient (P), etc.). To accomplish this, processor (52) executes an algorithm to calculate location coordinates of the distal end of the instrument from the position related signals of the coil(s) in the instrument.

Processor (52) uses software stored in a memory of processor (52) to calibrate and operate IGS navigation system (50). Such operation includes driving field generators (64), processing data from the instrument, processing data from operating controls (54), and driving display screen (56). In some implementations, operation may also include monitoring and enforcement of one or more safety features or functions of IGS navigation system (50). Processor (52) is further operable to provide video in real time via display screen (56), showing the position of the distal end of the instrument in relation to a video camera image of the patient's head (H), a CT scan image of the patient's head (H), and/or a computer generated three-dimensional model of the anatomy within and adjacent to the patient's nasal cavity. Display screen (56) may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, display screen (56) may provide images in accordance with at least some of the teachings of U.S. Pat. No. 10,463,242, entitled “Guidewire Navigation for Sinuplasty,” issued Nov. 5, 2019, the disclosure of which is incorporated by reference herein. In the event that the operator is also using an endoscope, the endoscopic image may also be provided on display screen (56).

The images provided through display screen (56) may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

II. Exemplary RF Ablation Surgical System Having Instrument with Expandable Ablation Member

In some instances, it may be desirable to provide an RF ablation instrument having one or more ablation members that are selectively actuatable between non-expanded and expanded states to facilitate effective and safe RF ablation of a nasal nerve, such as the posterior nasal nerve (40) as an alternative to a traditional vidian neurectomy procedure. Such ablation instruments may facilitate insertion into a nasal cavity to reach a target region while the ablation members are in a non-expanded state; and promote full contact with targeted tissue when the ablation members are in the expanded state. Each of the exemplary RF ablation instruments (110, 210, 310, 410) described below functions in such a manner. While the examples provided below are discussed in the context of posterior nasal nerve (40) ablation, RF ablation instruments (110, 210, 310, 410) may be used to ablate tissue in various other regions within the ear, nose, or throat of a patient. Other suitable ways in which RF ablation instruments (110, 210, 310, 410) may be used will be apparent to those skilled in the art in view of the teachings herein.

A. Exemplary RF Ablation Surgical System having Instrument with Electrodes Disposed on Inflatable Balloon

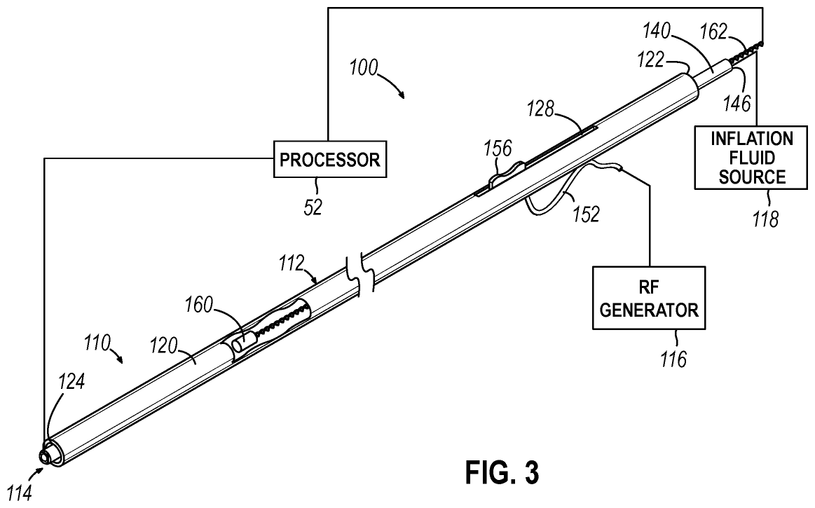

FIG. 3 shows an exemplary RF ablation surgical system (100) operable to ablate a nerve, such as the posterior nasal nerve (40), or other targeted region within the nasal cavity (10) of a patient with radio frequency (RF) energy. Surgical system (100) comprises a first exemplary RF ablation instrument (110) having a sheath (112), an ablation catheter (114) slidably disposed coaxially within sheath (112), and an RF generator (116) electrically coupled with at least one electrode of ablation catheter (114) to enable RF ablation of tissue (e.g., a nerve) positioned in electrical contact with the electrode, as described in greater detail below. System (100) of the present example further comprises an inflation fluid source (118) fluidly coupled with an inflation lumen of ablation catheter (114). Inflation fluid source (118) is configured to deliver an inflation fluid to a lumen of ablation catheter (114) to promote electrical coupling of the electrode with the tissue, as described in greater detail below.

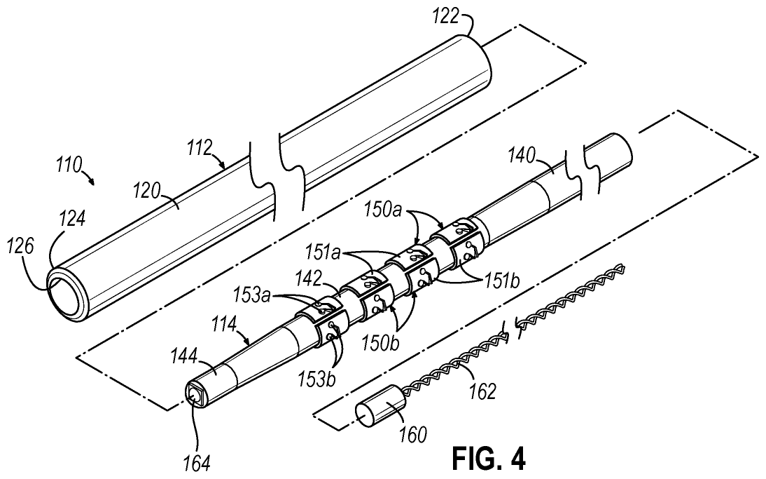

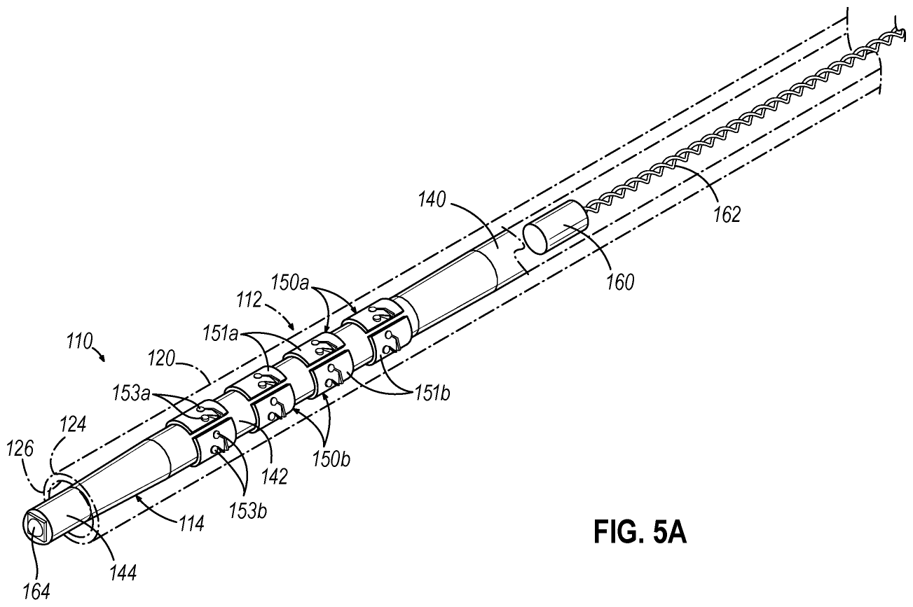

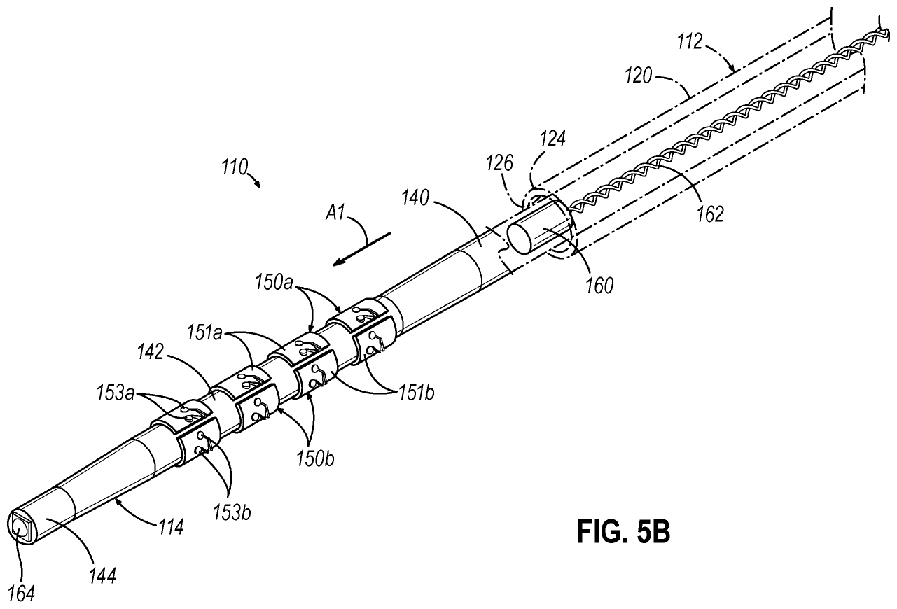

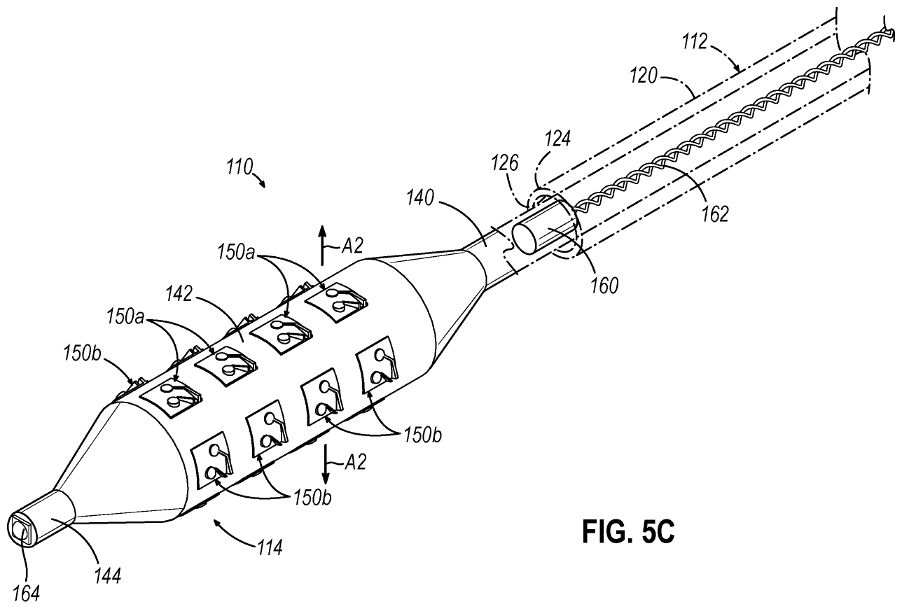

As shown in FIGS. 4-5C, sheath (112) of RF ablation instrument (110) includes an outer tube (120) having a proximal end (122), a distal end (124), and a tube lumen (126) extending therebetween. Tube lumen (126) is sized to slidably receive ablation catheter (114) therein, as described below. A proximal portion of outer tube (120) includes a first longitudinal slot (128) extending laterally through a first side of outer tube (120), and a second longitudinal slot (not shown) extending laterally through an opposed second side of outer tube (120). In some versions, a pull wire or other actuator may be coupled to a portion of sheath (112), such as distal end (124), to enable controlled lateral deflection or steering of sheath (112) during navigation of sheath (112) through the nasal cavity (10).

Ablation catheter (114) includes a proximal inner tube or elongate shaft (140) extending along a longitudinal axis, an expandable ablation member in the form of an inflatable balloon (142) extending distally from shaft (140), and a distal tip (144) extending distally from inflatable balloon (142) and defining a distal end of ablation catheter (114). Balloon (142) may be compliant or non-compliant, as may be desired. In the example shown, distal tip (144) is atraumatic to avoid inadvertently piercing tissue. In some versions, distal tip (144) may be substantially rigid to permit selective insertion of distal tip (144) into the turbinate (20, 22, 24) of a patient. In addition, some versions of distal tip (144) may be sharp such that distal tip (144) is configured to pierce tissue. A shaft lumen (146) extends longitudinally through shaft (140) and is in fluid communication with an internal cavity of balloon (142). As described below, shaft lumen (146) is configured to fluidly couple with inflation fluid source (118) to communicate inflation fluid from inflation fluid source (104) to balloon (142). Distal tip (144) of the present example extends coaxially with shaft (140) and outer tube (120).

Ablation catheter (114) further comprises a plurality of electrode assemblies (150a, 150b), including a plurality of active electrode assemblies (150a) and a plurality of return electrode assemblies (150b), disposed on an external surface of balloon (142). In the present example, each electrode assembly (150a, 150b) is in the form of a flex circuit. In particular, each electrode assembly (150a, 150b) of the present example includes a flexible substrate (151a, 151b) and electrode contacts (153a, 153b) disposed on substrate (151a, 151b). Electrode assemblies (150a, 150b) of the present example may each provide a relatively low profile that does not require substantial spatial accommodations around the external surface of balloon (142).

By way of example only, substrate (151a, 151b) may comprise polyimide, polyether ether ketone (PEEK), polyester, or any other suitable material. Substrates (151a, 151b) may be secured to balloon (142) in any suitable fashion, including but not limited to being secured via an adhesive. The flexibility of substrates (151a, 151b), in combination with the spacing between substrates (151a, 151b), may ensure that electrode assemblies (150a, 150b) do not unduly interfere with the balloon (142) transitioning between the non-expanded and expanded states.

Electrode contacts (153a, 153b) may be configured as a thin film and may formed of any suitable material or combination of materials, including but not limited to metallic conductive materials such as copper, gold, steel, aluminum, silver, nitinol, etc. and/or non-metallic conductive materials such as conducting polymers, silicides, graphite, etc. Electrode contacts (153a, 153b) may be secured to substrates (151a, 151b) using conventional circuit printing techniques, vapor deposition, or in any other suitable fashion as will be apparent to those skilled in the art in view of the teachings herein.

In the present example, electrode assemblies (150a, 150b) are configured to cooperate with each other to deliver RF energy from RF generator (116) to tissue positioned in electrical contact with electrode assemblies (150a, 150b), to thereby ablate the tissue with bipolar RF energy. Alternatively, electrode assemblies (150a, 150b) may be used to apply electroporation energy to tissue (e.g., to promote absorption of therapeutic agents, etc.). An electrical connector (152) (e.g., a wire) is affixed to a proximal portion of shaft (140) and is configured to electrically couple with RF generator (116) to deliver RF energy to electrode assemblies (150a, 150b) via a conductor such as one or more wires (not shown). Such a configuration may be configured and operable in accordance with any one or more of the teachings of U.S. Pub. No. 2020/0261149, entitled “Instrument for Endoscopic Posterior Nasal Nerve Ablation,” published Aug. 20, 2020, the disclosure of which is incorporated by reference herein.

In the example shown, electrode assemblies (150a, 150b) are arranged on the external surface of balloon (142) in an angularly alternating, longitudinally aligned pattern such that electrode assemblies (150a, 150b) are circumferentially arranged about the external surface of balloon (142) in alternating longitudinal rows of active electrode assemblies (150a) and return electrode assemblies (150b). It will be appreciated that electrode assemblies (150a, 150b) may be provided in any suitable number and positioned on the external surface of balloon (142) in any other suitable arrangement or pattern. For example, electrode assemblies (150a, 150b) may be arranged on the external surface of balloon (142) in a longitudinally alternating, angularly aligned pattern such that electrode assemblies (150a, 150b) are longitudinally arranged along the external surface of balloon (142) in alternating circumferential columns of active electrode assemblies (150a) and return electrode assemblies (150b). As another example, electrode assemblies (150a, 150b) may be arranged on the external surface of balloon (142) in an angularly alternating, longitudinally alternating pattern such that electrode assemblies (150a, 150b) are circumferentially arranged about the external surface of balloon (142) in alternating longitudinal rows of alternating active electrode assemblies (150a) and return electrode assemblies (150b) (e.g., in a “checkerboard” pattern). As another merely illustrative example, each electrode assembly (150a) may include a pair of electrode contacts (153a) that include an active electrode contact (153a) and a return electrode contact (153a), such that each electrode assembly (150a) alone may be operable to apply bipolar RF energy to tissue. Similarly, each electrode assembly (150b) may include a pair of electrode contacts (153b) that include an active electrode contact (153b) and a return electrode contact (153b), such that each electrode assembly (150b) alone may be operable to apply bipolar RF energy to tissue.

Regardless of the particular arrangement of electrode assemblies (150a, 150b) on balloon (142), electrode contacts (153a, 153b) may be configured to cooperate with each other to treat tissue with bipolar RF energy. Such a configuration may advantageously provide sufficient energy levels needed for effective RF ablation of the posterior nasal nerve (40) or other targeted anatomical regions within the ear, nose, or throat. In some other versions, balloon (142) may be provided with a single electrode assembly (150a, 150b), such as a single active electrode assembly (150a), which may be configured to cooperate with an RF ground pad (not shown), placed on or under the patient, to apply monopolar RF energy to tissue. Similarly, balloon (142) may include a plurality of electrode assemblies (150a, 150b) that are all operable to provide just one single pole of RF energy, such that two or more electrode assemblies (150a, 150b) may cooperate with a ground pad to apply monopolar RF energy to tissue. Some versions of RF generator (116) may be operable to toggle between bipolar and monopolar modalities, such that the same balloon (142) and corresponding electrode assemblies (150a, 150b) may be operable to apply bipolar or monopolar RF energy to tissue, based on the selection of an operator of instrument (110).

Ablation catheter (114) further comprises a projection in the form of an elongate tab (156) projecting laterally outwardly from a proximal portion of shaft (140) generally in alignment with and opposed from electrical connector (152). Tab (156) is operable as an advancer to selectively actuate ablation catheter (114) longitudinally relative to sheath (112). In that regard, ablation catheter (114) is slidably housed within outer tube (120) of sheath (112) such that advancer tab (156) projects laterally through first longitudinal slot (128), and such that electrical connector (152) projects laterally through the opposing second longitudinal slot. In some other versions, electrical connector (152) passes out through proximal end (122) of outer tube (120) of sheath (112), such that electrical connector (152) need not necessarily project laterally through a laterally presented, longitudinally extending slot in outer tube (120) of sheath (112). Advancer tab (156) and electrical connector (152) are fixed to shaft (140) such that advancer tab (156) slides longitudinally within first longitudinal slot (128) and electrical connector (152) simultaneously slides longitudinally within the second longitudinal slot as shaft (140) translates through tube lumen (126). Advancer tab (156) may be formed of a non-conductive material or otherwise be electrically insulated from electrical connector (152) and electrode assemblies (150a, 150b). By way of example only, advancer tab (156) may be configured and operable in accordance with at least some of the teachings of U.S. Pub. No. 2020/0315697, entitled “Needle Instrument for Posterior Nasal Neurectomy Ablation,” published Oct. 8, 2020, the disclosure of which is incorporated by reference herein. Alternatively, any other suitable kind of actuator may be used to drive longitudinal translation of ablation catheter (114) relative to sheath (112).

In the example shown, ablation catheter (114) further includes a navigation sensor (160) that is responsive to positioning within the alternating magnetic fields generated by field generators (64) like that described above with reference to FIG. 2 to help guide maneuvering and otherwise manipulating RF ablation instrument (110) within the nasal cavity (10). Navigation sensor (160) of the present version is fixedly disposed within shaft lumen (146) at or near an interface between shaft (140) and balloon (142) (e.g., at a distal end of shaft (140) proximal of balloon (142)), and is operatively coupled to processor (52) via one or more wires (162). Ablation catheter (114) also includes a distally-facing camera (164) fixedly disposed at distal tip (144) for visualizing a patient's anatomy distal of distal tip (144) to help guide maneuvering and otherwise manipulating RF ablation instrument (110) within the nasal cavity (10). Camera (164) may be operatively coupled to processor (52) via one or more wires (not shown) for displaying images (e.g., still or video) captured by camera (164) via display screen (56).

Additionally, in some versions, ablation catheter (114) may further include one or more tissue sensors operable to sense a condition of the tissue (e.g., a nerve) being ablated by electrode assemblies (150a, 150b). Each such tissue sensor may communicate a signal to processor (52) indicating the sensed condition. In response to receiving the signal, processor (52) may then regulate (e.g., deactivate) the RF ablation energy being delivered to active electrode assemblies (150a) from RF generator (116), and/or provide an indication to the operator informing of the sensed tissue condition. In some versions, such a tissue sensor may comprise a thermocouple operable to measure a temperature of the target tissue during ablation. In other versions, such a tissue sensor may comprise a pair of detection electrodes operable to deliver a low power RF signal to the target tissue to measure an electrical impedance of the tissue during ablation. In some such versions, such detection electrodes may be provided separately from electrode assemblies (150a, 150b). In other such versions, one or more ablation contacts (153a, 153b) may be operable as both an ablation electrode and as a detection electrode. In either configuration, the low power RF signal may be delivered to the target tissue simultaneously or in rapidly alternating fashion with the high-power RF ablation energy delivered by ablation contacts (153a, 153b). While the target tissue remains substantially intact and unablated, the low power RF signal may pass freely through the tissue with a relatively low impedance. As ablation of the tissue progresses, the detection electrodes may detect a corresponding increase in impedance of the tissue, which is communicated to processor (52).

In some versions, each electrode assembly (150a, 150b) may be activated independently to enable adjusting one or more ablation regions of tissue. For example, a first adjacent pair of active and return electrode assemblies (150a, 150b) on one side of balloon (142) may be selectively activated while the remaining electrode assemblies (150a, 150b) remain deactivated to selectively ablate a first region of tissue extending between the first adjacent pair of electrode assemblies (150a, 150b). As another example, a second adjacent pair of active and return electrode assemblies (150a, 150b) on another side of balloon (142) may be selectively activated while the first adjacent pair of electrode assemblies (150a, 150b) is activated to simultaneously ablate a second region of tissue extending between the second adjacent pair of electrode assemblies (150a, 150b) during ablation of the first region of tissue. As described above, such activation may be performed automatically by processor (52), such as in response to feedback received by processor (52) from the tissue sensors and/or from navigation sensor (160) to ensure proper ablation of the desired target tissue. In addition, or alternatively, such activation may be performed manually by the operator, such as based on images or other information displayed on display screen (56).

As shown in FIGS. 5A and 5B, ablation catheter (114) is selectively actuatable via advancer tab (156) between a proximal retracted position (FIG. 5A) and a distal extended position (FIG. 5B) relative to outer tube (120) of sheath (112), as indicated by first arrow (Al) in FIG. 5B. In the exemplary proximal retracted position of FIG. 5A, advancer tab (156) and electrical connector (152) may be disposed at the proximal ends of first longitudinal slot (128) and the second longitudinal slot, respectively, distal tip (144) is at least partially concealed within the distal end of outer tube (120) (e.g., with camera (164) extending distally beyond the distal end of outer tube (120)), and balloon (142) is housed coaxially within outer tube (120) in a non-expanded state. In this regard, tube lumen (126) may have a cross dimension sized to constrict balloon (142) to the non-expanded state. Thus, outer tube (120) may prevent balloon (142) from radially expanding or otherwise restrict radial expansion of balloon (142) when ablation catheter (114) is in the proximal retracted position. In the exemplary distal extended position of FIG. 5B, advancer tab (156) and electrical connector (152) may be disposed at the distal ends of first longitudinal slot (128) and the second longitudinal slot, respectively, and distal tip (144) and balloon (142) are each exposed from outer tube (120) so as to extend distally beyond the distal end of outer tube (120), with balloon (142) remaining in the non-expanded state. Thus, outer tube (120) may permit balloon (142) to radially expand when ablation catheter (114) is in the distal extended position.

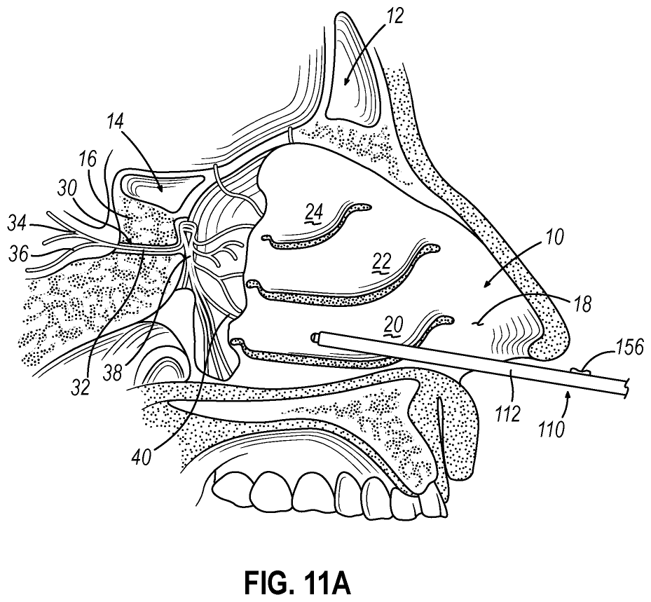

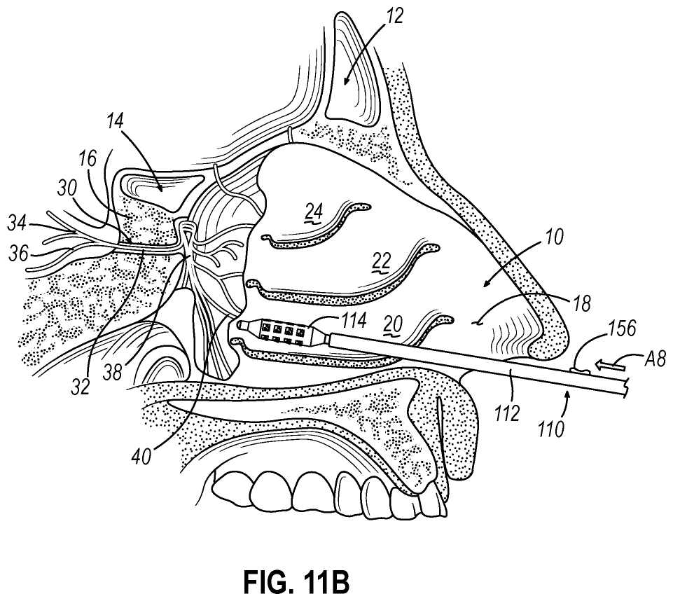

As shown in FIGS. 5B and 5C, when ablation catheter (114) is in the distal extended position, balloon (142) is selectively radially expandable via inflation fluid delivered thereto from inflation fluid source (118) and shaft lumen (146) from the non-expanded state (FIG. 5B) to an expanded state (FIG. 5C) so as to extend radially outwardly beyond shaft (140), as indicated by second arrows (A2) in FIG. 5C. As described in greater detail below in connection with FIGS. 11A-11B, ablation catheter (114) is configured to be actuated from the proximal retracted position to the distal extended position and balloon (142) is configured to be subsequently expanded from the non-expanded state to the expanded state to urge electrode contacts (153a, 153b) into electrical contact with the target tissue of a patient following insertion of the distal end of RF ablation instrument (110) into the nasal cavity (10).

B. Alternative Instrument with Electrodes Disposed on Inflatable Balloon and Sheath Camera

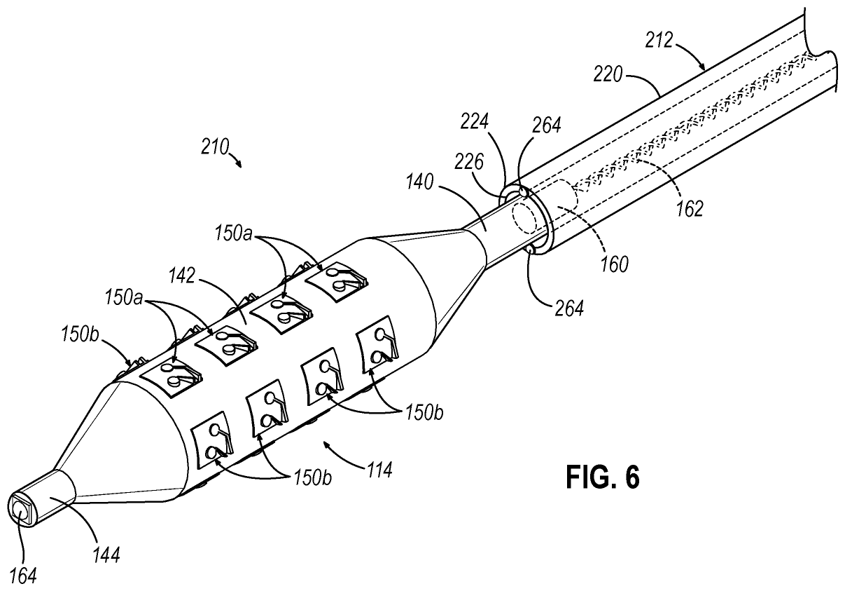

In some instances, it may be desirable to provide an RF ablation instrument for use with system (100) having additional visualization capabilities as compared to RF ablation instrument (110). FIG. 6 shows a second exemplary RF ablation instrument (210) having such capabilities. RF ablation instrument (210) is substantially similar to RF ablation instrument (110) except as otherwise described herein.

RF ablation instrument (210) includes a sheath (212) and ablation catheter (114) slidably disposed coaxially within sheath (212). Sheath (212) of RF ablation instrument (210) includes an outer tube (220) having a proximal end (not shown), a distal end (224), and a tube lumen (226) extending therebetween. In the example shown, sheath (212) further includes one or more (e.g., two) distally-facing cameras (264) fixedly disposed at distal end (224) of outer tube (220) for visualizing a patient's anatomy distal of distal end (224) to help guide maneuvering and otherwise manipulating RF ablation instrument (210) within the nasal cavity (10). In addition or alternatively, cameras (264) may be used for visualizing balloon (142) of ablation catheter (114), such as for visually assessing whether balloon (142) is in the non-expanded or expanded state, and/or for visualizing electrical contact between one or more electrode assemblies (150a, 150b) and the target tissue, and the resulting ablation of the target tissue. Cameras (264) may be operatively coupled to processor (52) via one or more wires (not shown) for displaying images (e.g., still or video) captured by cameras (264) via display screen (56).

C. Alternative Instrument with Electrodes Disposed on Inflatable Balloon and Adjustable Camera

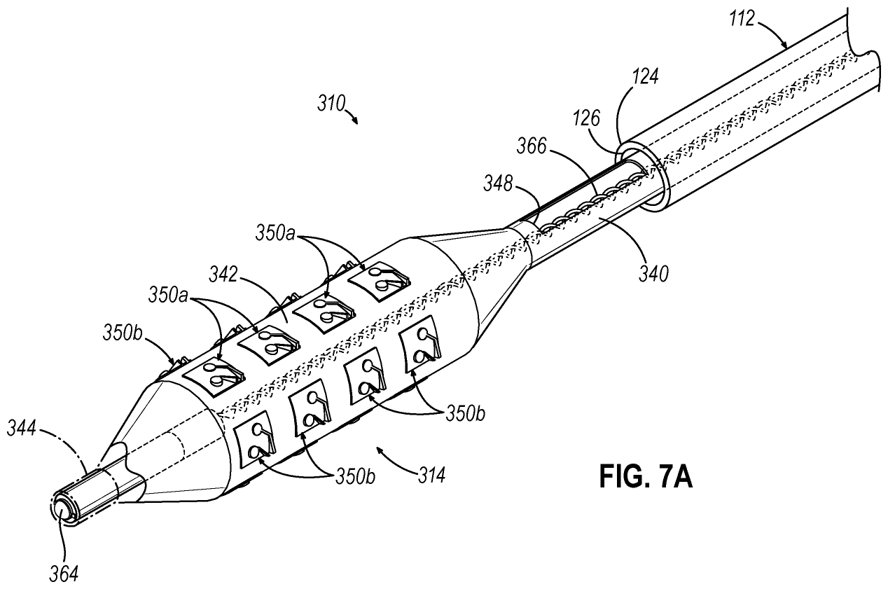

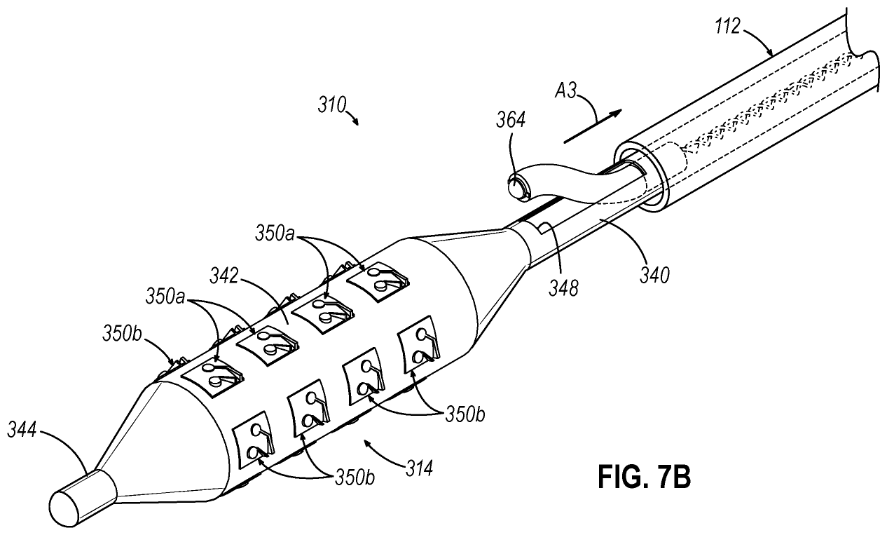

In some instances, it may be desirable to provide an RF ablation instrument for use with system (100) having additional visualization capabilities as compared to RF ablation instrument (210). FIGS. 7A-7B show a third exemplary RF ablation instrument (310) having such capabilities. RF ablation instrument (310) is substantially similar to RF ablation instrument (110) except as otherwise described herein.

RF ablation instrument (310) includes sheath (112) and an ablation catheter (314) slidably disposed coaxially within sheath (112). Ablation catheter (314) includes a proximal inner tube or elongate shaft (340) extending along a longitudinal axis, an expandable ablation member in the form of an inflatable balloon (342) extending distally from shaft (340), and a distal tip (344) extending distally from inflatable balloon (342) and defining a distal end of ablation catheter (314). In some versions, distal tip (344) is closed and formed of a transparent material, thereby allowing visualization through distal tip (344). In some other versions, distal tip (344) is open. A shaft lumen (not shown) extends longitudinally through shaft (340) and is in fluid communication with an internal cavity of balloon (342). A distal portion of shaft (340) includes a longitudinal slot (348) extending laterally through a side of shaft (340). Slot (348) is proximal to balloon (342). Ablation catheter (314) further comprises a plurality of active electrode assemblies (350a) and a plurality of return electrode assemblies (350b) disposed on an external surface of balloon (342). Electrode assemblies (350a, 350b) may be configured and operable just like electrode assemblies (150a, 150b) described above.

In the example shown, RF ablation instrument (310) further includes a distally-facing camera (364) for selectively visualizing a patient's anatomy distal of distal tip (344) to help guide maneuvering and otherwise manipulating RF ablation instrument (310) within the nasal cavity (10), and for selectively visualizing balloon (342) of ablation catheter (314), such as for visually assessing whether balloon (342) is in the non-expanded or expanded state, and/or for visualizing electrical contact between one or more electrode assemblies (350a, 350b) on balloon (342) and the target tissue, and the resulting ablation of the target tissue. Camera (364) may be operatively coupled to processor (52) via one or more wires (366) for displaying images (e.g., still or video) captured by camera (364) via display screen (56).

In this regard, camera (364) is selectively actuatable between a distal extended position (FIG. 7A) and a proximal retracted position (FIG. 7B) relative to balloon (342) of ablation catheter (314), as indicated by third arrow (A3) in FIG. 7B. In the exemplary distal extended position of FIG. 7A, camera (364) is selectively disposed at distal tip (344) so as to visualize a patient's anatomy distal of distal tip (344). For example, as noted above, distal tip (344) may be transparent to permit camera (364) to visualize the patient's anatomy through distal tip (344) while distal tip (344) encloses camera (364). Alternatively, in versions where distal tip (344) is open, camera (364) may be advanced through the opening at distal tip (344) or may be otherwise positioned to provide visualization through the opening of distal tip (344).

In the exemplary proximal retracted position of FIG. 7B, camera (364) selectively extends through longitudinal slot (348) so as to visualize balloon (342) of ablation catheter (314). In this position, the line of sight for camera (364) is parallel with, yet laterally offset from, the longitudinal axis of ablation catheter (314). In some versions, camera (364) may be resiliently biased radially outwardly through longitudinal slot (348), such that camera (364) is configured to automatically extend through longitudinal slot (348) when longitudinally aligned therewith. For example, a nitinol wire (not shown) may extend along at least a portion of camera (364) and/or wires (366) to thereby resiliently bias camera (364) radially outwardly in manner that allows camera (364) to continue facing distally. In this manner, camera (364) may initially be in the distal extended position during navigation of RF ablation instrument (310) through the nasal cavity (10), and may be subsequently actuated to the proximal retracted position by translating camera (364) proximally relative to balloon (342) into longitudinal alignment with longitudinal slot (348), thereby allowing camera (364) to automatically extend radially outwardly through longitudinal slot (348). In some other versions, a pull wire or other actuator may be manipulated by the operator to selectively control and drive the lateral deflection of camera (364) out through longitudinal slot (348) to achieve the configuration shown in FIG. 7B.

D. Alternative Instrument with Expandable Mesh Electrodes

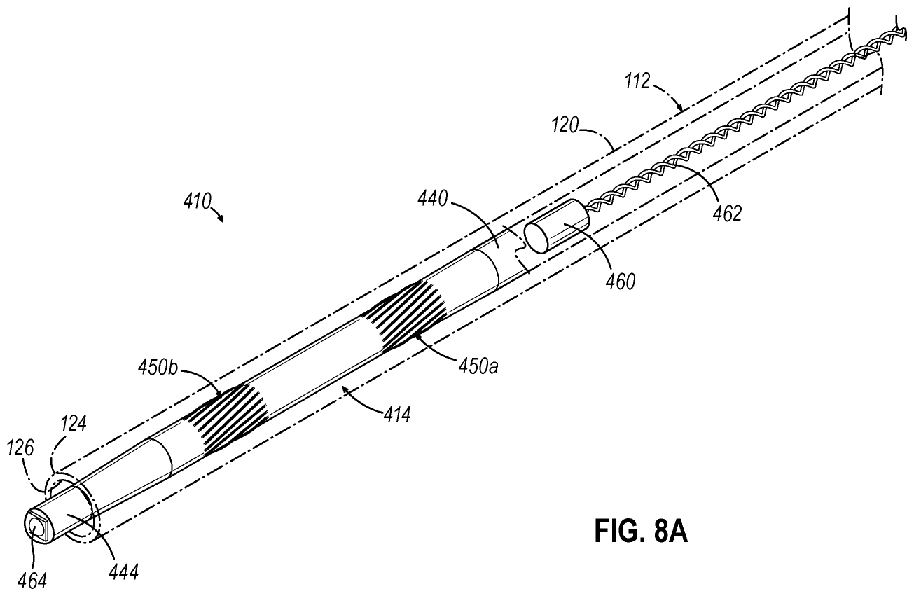

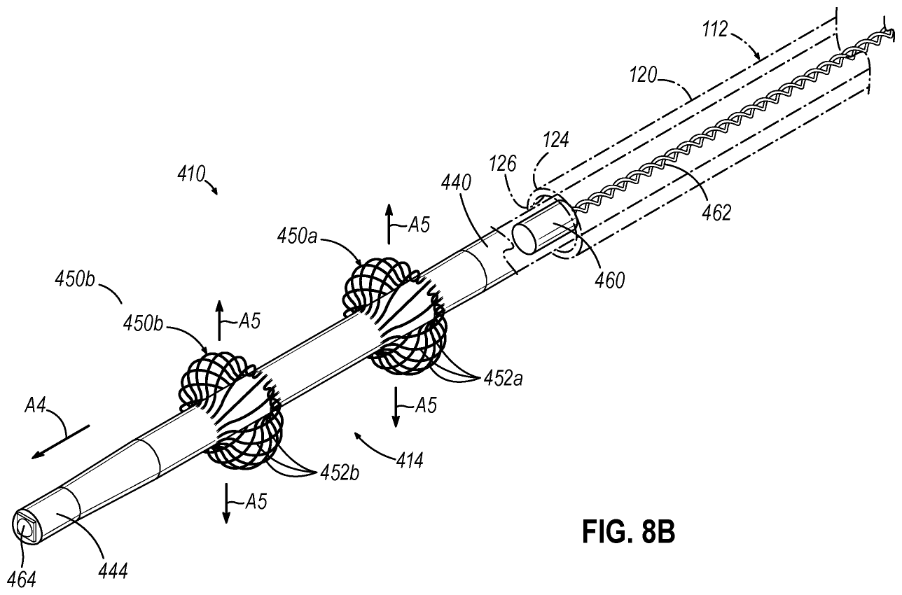

In some instances, it may be desirable to provide an RF ablation instrument for use with system (100) having an alternative expandable ablation member from that of RF ablation instrument (110). FIGS. 8A-8B show a fourth exemplary RF ablation instrument (410) having such a configuration. RF ablation instrument (410) is substantially similar to RF ablation instrument (110) except as otherwise described herein.

RF ablation instrument (410) includes sheath (112) and an ablation catheter (414) slidably disposed coaxially within sheath (112). Ablation catheter (414) includes a proximal inner elongate shaft (440) extending along a longitudinal axis, and a distal tip (444) extending distally from shaft (440) and defining a distal end of ablation catheter (414). In the example shown, distal tip (444) is atraumatic to avoid inadvertently piercing tissue. In some versions, distal tip (444) may be substantially rigid to permit selective insertion of distal tip (444) into the turbinate (20, 22, 24) of a patient. In addition, some versions of distal tip (444) may be sharp such that distal tip (444) is configured to pierce tissue.

In the example shown, ablation catheter (414) further includes a pair of expandable ablation members in the form of resiliently biased electrode assemblies (450a, 450b), including an active electrode assembly (450a) and a return electrode assembly (450b), disposed on shaft (440). Electrode assembly (450a) includes a plurality of wire members (452a) that are angularly spaced apart from each other about the circumference of shaft (440). Similarly, electrode assembly (450b) includes a plurality of wire members (452b) that are angularly spaced apart from each other about the circumference of shaft (440). Wire members (452a, 452b) are resiliently biased to bow radially outwardly to achieve the mesh or basket-like configuration shown in FIG. 8B. Wire members (452a, 452b) are nevertheless flexible enough to deflect inwardly and thereby be contained within sheath (112) as shown in FIG. 8A. In some versions, wire members (452a, 452b) are formed of a material that is resilient and electrically conductive (e.g., nitinol, spring steel, etc.). In addition, or in the alternative, each wire member (452a, 452b) may include a resilient structural support that is conductive or non-conductive, with a conductive material (e.g., copper, gold, steel, aluminum, silver, conducting polymers, silicides, graphite, etc.) deposited onto or otherwise secured to the resilient structural support.

In the example shown, electrode assemblies (450a, 450b) each have a generally annular cross-sectional shape such that each electrode assembly (450a, 450b) is circumferentially wrapped around shaft (440) and encircles or angularly surrounds the longitudinal axis of shaft (440). Electrode assemblies (450a, 450b) are configured to cooperate with each other to deliver RF energy from RF generator (116) to tissue positioned in electrical contact with electrode assemblies (450a, 450b), to thereby ablate the tissue with bipolar RF energy. Alternatively, electrode assemblies (450a, 450b) may be used to apply electroporation energy to tissue (e.g., to promote absorption of therapeutic agents, etc.). Electrode assemblies (450a, 450b) may be electrically coupled with an electrical connector (not shown) affixed to a proximal portion of shaft (440) and configured to electrically couple with RF generator (116) to deliver RF energy to electrode assemblies (450a, 450b) via a conductor such as one or more wires (not shown).

In the example shown, electrode assemblies (450a, 450b) are arranged on shaft (440) such that active electrode assembly (450a) is in a relatively proximal position and return electrode assembly (450b) is in a relatively distal position. It will be appreciated that electrode assemblies (450a, 450b) may be provided in any suitable number and positioned on shaft (140) in any other suitable arrangement or pattern. For example, electrode assemblies (450a, 450b) may be arranged on shaft (440) such that active electrode assembly (450a) is in a relatively distal position and return electrode assembly (450b) is in a relatively proximal position.

In some versions, distal tip (444) may be formed of an electrically conductive material, such that distal tip (444) itself defines another electrode. Similar to electrode assemblies (450a, 450b), distal tip (444) may be electrically coupled with the electrical connector configured to electrically couple with RF generator (116) to deliver RF energy to distal tip (444) via a conductor such as one or more wires (not shown). In the present version, distal tip (444) may define an active electrode such that return electrode assembly (450b) may be positioned between two active electrodes (444, 450a). In other versions, such as those in which active electrode assembly (450a) is in a relatively distal position and return electrode assembly (450b) is in a relatively proximal position, distal tip (444) may define a return electrode. By defining an electrode itself, distal tip (444) may be suitable for insertion into the turbinate (20, 22, 24) of a patient and subsequent ablation of the turbinate (20, 22, 24) via cooperation with return electrode assembly (450b), for example. In some other versions, distal tip (444) does not define or otherwise provide an electrode, such that the only electrodes are provided by electrode assemblies (450a, 450b).

As described above, RF ablation instrument (410) of the present example includes a plurality of electrode assemblies (450a, 450b) disposed on shaft (440) and an electrode defined by distal tip (444) configured to cooperate with each other to treat tissue with bipolar RF energy. Such a configuration advantageously provides sufficient energy levels needed for effective RF ablation of the posterior nasal nerve (40). However, it will be appreciated that in other versions, shaft (440) may be provided with a single electrode assembly (450a, 450b), such as a single active electrode assembly (450a), which may be configured to cooperate with an RF ground pad (not shown) to treat various types of tissue with monopolar RF energy.

In the example shown, ablation catheter (414) further includes a navigation sensor (460) fixedly disposed within shaft (440) and operatively coupled to processor (52) via one or more wires (462), and a distally-facing camera (464) fixedly disposed at distal tip (144). While camera (464) of the present version is shown fixedly disposed at distal tip (144), ablation catheter (414) may additionally or alternatively include a camera that is selectively actuatable between a distal extended position (e.g., to visualize a patient's anatomy distal of distal tip (444)) and a proximal retracted position (e.g., to visualize expandable electrode assemblies (450a, 450b)) relative to expandable electrode assemblies (450a, 450b) of ablation catheter (414), similar to camera (364) described above with reference to FIGS. 7A and 7B. Additionally, in some versions, ablation catheter (414) may further include one or more tissue sensors operable to sense a condition of the tissue (e.g., a nerve) being ablated by electrodes (444, 450a, 450b).

In some versions, each electrode (444, 450a, 450b) may be activated independently to enable adjusting one or more ablation regions of tissue. For example, expandable electrode assemblies (450a, 450b) may be selectively activated while the electrode defined by distal tip (444) remains deactivated to selectively ablate a first region of tissue extending between expandable electrode assemblies (450a, 450b). As another example, the electrode defined by distal tip (444) may be selectively activated while expandable electrode assemblies (450a, 450b) are activated to simultaneously ablate a second region of tissue extending between return expandable electrode assembly (450b) and distal tip (444) during ablation of the first region of tissue. As described above, such activation may be performed automatically by processor (52), such as in response to feedback received by processor (52) from the tissue sensors and/or from navigation sensor (460) to ensure proper ablation of the desired target tissue. In addition, or alternatively, such activation may be performed manually by the operator, such as based on images or other information displayed on display screen (56).

As shown in FIGS. 8A and 8B, ablation catheter (414) is selectively actuatable (e.g., via an advancer tab) between a proximal retracted position (FIG. 8A) and a distal extended position (FIG. 8B) relative to outer tube (120) of sheath (112), as indicated by fourth arrow (A4) in FIG. 8B. In the exemplary proximal retracted position of FIG. 8A, distal tip (444) is at least partially concealed within the distal end of outer tube (120), and electrode assemblies (450a, 450b) are each housed coaxially within outer tube (120) in a non-expanded state. In this regard, tube lumen (126) may have a cross dimension sized to constrict electrode assemblies (450a, 450b) to the non-expanded state. Thus, outer tube (120) may radially compress electrode assemblies (450a, 450b) and prevent electrode assemblies (450a, 450b) from radially expanding or otherwise restrict radial expansion of electrode assemblies (450a, 450b) when ablation catheter (414) is in the proximal retracted position. In the exemplary distal extended position of FIG. 8B, distal tip (444) and electrode assemblies (450a, 450b) are each exposed from outer tube (120) so as to extend distally beyond the distal end of outer tube (120). Thus, outer tube (120) may permit electrode assemblies (150a, 150b) to resiliently radially expand in response to ablation catheter (414) transitioning to the distal extended position.

As shown in FIG. 8B, electrode assemblies (450a, 450b) are each automatically radially expandable from the non-expanded state to the expanded state so as to extend radially outwardly beyond shaft (440) in response to actuation of ablation catheter (414) to the distal extended position, as indicated by fifth arrows (A5) in FIG. 8B. Thus, ablation catheter (414) is configured to be actuated from the proximal retracted position to the distal extended position and in response thereto electrode assemblies (450a, 450b) are configured to be automatically expanded from the non-expanded state to the expanded state to urge electrode assemblies (450a, 450b) into electrical contact with the target tissue of a patient following insertion of the distal end of RF ablation instrument (410) into the nasal cavity (10).

As noted above, electrode assemblies (450a, 450b) are configured such that wire members (452a) of electrode assembly (450a) are configured to serve as active electrodes for application of bipolar RF energy; while wire members (452b) of electrode assembly (450b) are configured to serve as return electrodes for application of bipolar RF energy. Thus, the bipolar RF energy may be applied to contacted tissue that is positioned between electrode assembly (450a) and electrode assembly (450b). In some versions, electrode assembly (450a) may be configured such that wire members (452a) are operable to apply bipolar RF energy to tissue. For instance, electrode assembly (450a) may include some wire members (452a) that are configured to serve as active electrodes and other wire members (452a) that are configured to serve as return electrodes, with these active/return wire members (452a) being positioned in any suitable relationship with each other. For instance, the active electrode wire members (452a) may span approximately 180 degrees about the circumference of shaft (440); while the return electrode wire members (452a) may span the other approximately 180 degrees about the circumference of shaft (440). As another merely illustrative example, the active and return electrode wire members (452a) may be positioned in an angularly alternating fashion about the circumference of shaft (440). Other suitable ways in which electrode assembly (450a) may be configured to apply bipolar RF energy (e.g., without electrode assembly (450b) necessarily playing any role in such application of bipolar RF energy) will be apparent to those skilled in the art in view of the teachings herein. Of course, electrode assembly (450b) may also be configured to apply bipolar RF energy (e.g., without electrode assembly (450a) necessarily playing any role in such application of bipolar RF energy) in accordance with the above teachings.

In versions where electrode assembly (450a) and/or electrode assembly (450b) is/are capable of applying bipolar RF energy independently, and electrode assemblies (450a, 450b) are also configured to cooperate with each other to apply bipolar RF energy, a control module (not shown) may enable the operator to select a preferred mode of operation. In other words, the control module may allow the operator to select between a first mode, in which electrode assembly (450a) and/or electrode assembly (450b) applies bipolar RF energy to tissue independently of the other electrode assembly (450a, 450b); and a second mode, in which electrode assemblies (450a, 450b) cooperate with each other to apply bipolar RF energy to tissue. In addition, or in the alternative, such a control module may allow the operator to select a mode where electrode assembly (450a) and/or electrode assembly (450b) cooperates with a ground pad to apply monopolar RF energy to tissue.

E. Alternative Ablation Catheter with Expandable Mesh Electrode at Distal Tip

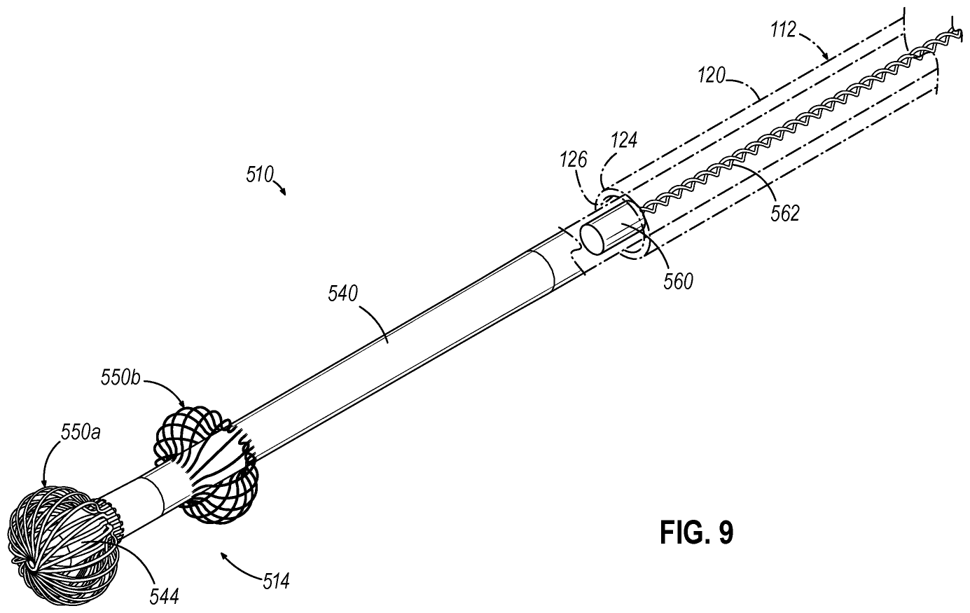

In some instances, it may be desirable to provide an RF ablation instrument for use with system (100) having an alternative expandable electrode arrangement from that of RF ablation instrument (410). FIG. 9 shows a fifth exemplary RF ablation instrument (510) having such a configuration. RF ablation instrument (510) is substantially similar to RF ablation instrument (410) except as otherwise described herein.

RF ablation instrument (510) includes sheath (112) and an ablation catheter (514) slidably disposed coaxially within sheath (112). Ablation catheter (514) includes a proximal inner elongate shaft (540) extending along a longitudinal axis, and a distal tip (544) extending distally from shaft (540). Ablation catheter (514) further includes a pair of expandable ablation members in the form of resiliently biased electrode assemblies (550a, 550b), including an active electrode assembly (550a) and a return electrode assembly (550b), disposed on shaft (540). Ablation catheter (514) also includes a navigation sensor (560) fixedly disposed within shaft (540) and operatively coupled to processor (52) via one or more wires (562).

In the example shown, return electrode assembly (550b) is in a relatively proximal position and active electrode assembly (550a) is positioned at distal tip (544) such that a distalmost portion of active electrode assembly (550a) is distal of distal tip (544) and thereby defines a distal end of ablation catheter (514). In this manner, active electrode assembly (550a) may be capable of contacting tissue distal of distal tip (544) in addition to tissue radially outward of shaft (540) to promote ablation of tissue distal of distal tip (544). In other versions, active electrode assembly (550a) may be in a relatively proximal position and return electrode assembly (550b) may be positioned at distal tip (544) such that a distalmost portion of return electrode assembly (550b) is distal of distal tip (544) and thereby defines a distal end of ablation catheter (514). Aside from the different positioning of electrode assemblies (550a, 550b) in RF ablation instrument (510), electrode assemblies (550a, 550b) of RF ablation instrument (510) may be configured and operable like electrode assemblies (450a, 450b) of RF ablation instrument (410) described above.

F. Alternative Ablation Catheter with Translatable Expandable Mesh Electrodes

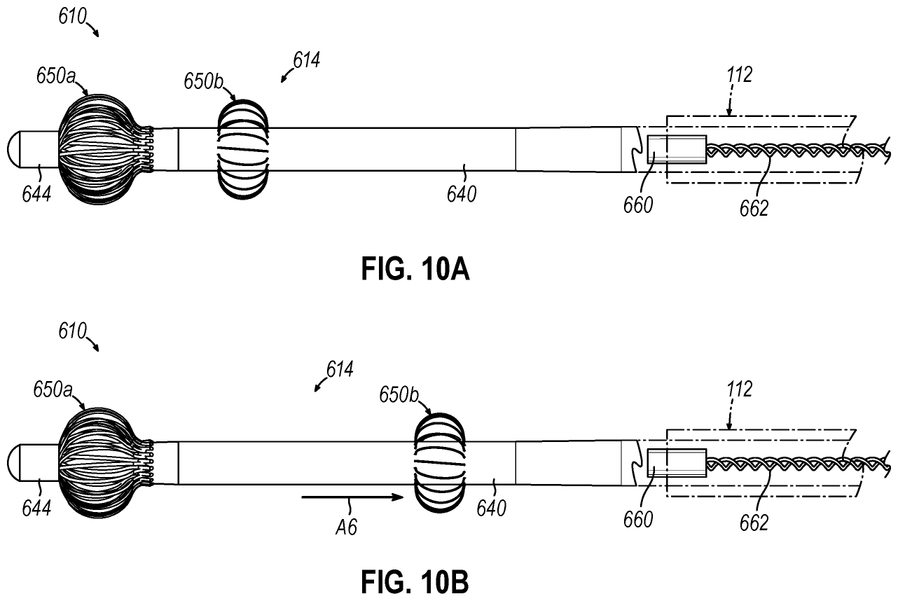

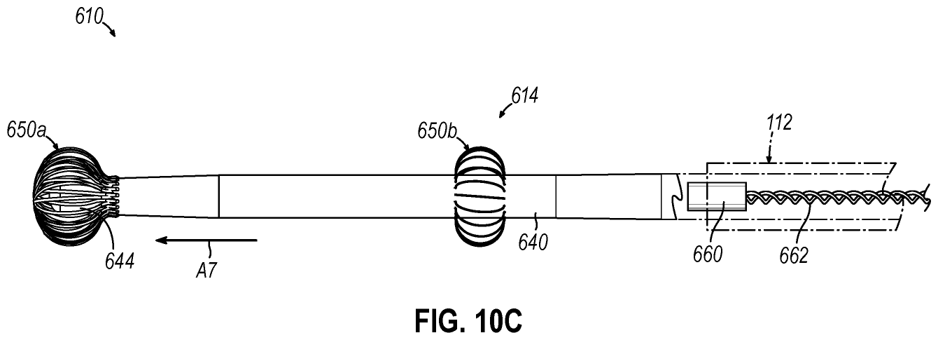

In some instances, it may be desirable to provide an RF ablation instrument for use with system (100) having an adjustable expandable electrode arrangement. FIGS. 10A-10C show a sixth exemplary RF ablation instrument (610) having such a configuration. RF ablation instrument (610) is substantially similar to RF ablation instrument (410) except as otherwise described herein.

RF ablation instrument (610) includes sheath (112) and an ablation catheter (614) slidably disposed coaxially within sheath (112). Ablation catheter (614) includes a proximal inner elongate shaft (640) extending along a longitudinal axis, and a distal tip (644) extending distally from shaft (640). Ablation catheter (614) further includes a pair of expandable ablation members in the form of resiliently biased electrode assemblies (650a, 650b), including an active electrode assembly (650a) and a return electrode assembly (650b), disposed on shaft (640). Ablation catheter (614) also includes a navigation sensor (660) fixedly disposed within shaft (640) and operatively coupled to processor (52) via one or more wires (662).

In the example shown, electrode assemblies (650a, 650b) are selectively longitudinally translatable relative to each other along shaft (640), as indicated by sixth arrow (A6) in FIG. 10B and seventh arrow (A7) in FIG. 10C, with return electrode assembly (650b) being in a relatively proximal position and active electrode assembly (650a) being in a relatively distal position. For example, return electrode assembly (650b) may be longitudinally translated proximally away from active electrode assembly (650a) from a first proximal position (FIG. 10A) to a second proximal position (FIGS. 10B and 10C). Likewise, active electrode assembly (650a) may be longitudinally translated distally away from return electrode assembly (650b) from a first distal position (FIGS. 10A and 10B) to a second distal position (FIG. 10C). Such longitudinal translation of electrode assemblies (650a, 650b) relative to each other may enable adjusting an ablation region of tissue. In other versions, active electrode assembly (650a) may be in a relatively proximal position and return electrode assembly (650b) may be in a relatively distal position. The longitudinal translation of electrode assemblies (650a, 650b) may be actuated via corresponding sliders or other actuation inputs on a handle assembly (not shown). Various suitable ways in which longitudinal translation of electrode assemblies (650a, 650b) may be actuated will be apparent to those skilled in the art in view of the teachings herein. Aside from the different positionability of electrode assemblies (650a, 650b) in RF ablation instrument (510), electrode assemblies (650a, 650b) of RF ablation instrument (610) may be configured and operable like electrode assemblies (450a, 450b) of RF ablation instrument (410) described above.

III. Exemplary Method of Ablating Posterior Nasal Nerve

Having described exemplary features of RF ablation surgical system (100) and RF ablation instruments (110, 210, 310, 410) above, an exemplary method of performing an ablation on a posterior nasal nerve (40) of a patient with system (100) will now be described in connection with FIGS. 11A-11B. While the exemplary method is showing being performed with RF ablation instrument (110), it will be appreciated that similar methods may be performed using RF ablation instruments (210, 310, 410). Additionally, while surgical system (100) is shown and described for treating a posterior nasal nerve, it will be appreciated that surgical system (100) may be employed in various other surgical applications for ablating other nerves or anatomical structures within the nasal cavity (10), or for ablating tissues in various other anatomical regions of a patient. For instance, the teachings herein may be combined with at least some of the teachings of U.S. Pat. Pub. No. 2019/0374280, entitled “Apparatus and Method for Performing Vidian Neurectomy Procedure,” published Dec. 12, 2019, the disclosure of which is incorporated by reference herein.

As shown in FIG. 11A, the distal end of RF ablation instrument (110) is inserted into the nasal cavity (10) and is toward the posterior ends of the inferior and middle turbinates (20, 22), which may be performed under visualization provided by camera (164) or by a separate endoscope (not shown), for example. Upon reaching a target site of the nasal wall (18) in which a target portion of the posterior nasal nerve (40) resides, the operator advances advancer tab (156) distally to thereby extend ablation catheter (114), as indicated by eighth arrow (A8) in FIG. 11B, and subsequently inflates balloon (142), as shown in FIG. 11B, to urge electrode assemblies (150a, 150b) into electrical contact with a target portion of posterior nasal nerve (40). Electrode assemblies (150a, 150b) are then energized with bipolar RF energy to thereby ablate the targeted portion of posterior nasal nerve (40).

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A surgical instrument comprising: (a) a sheath configured to be inserted into a cavity of a patient's head; and (b) an ablation catheter disposed within the sheath, wherein the ablation catheter extends along a longitudinal axis and includes at least one expandable ablation member configured to selectively radially expand outwardly from a non-expanded state to an expanded state for selectively ablating tissue within the patient's head, wherein the ablation catheter is selectively translatable relative to the sheath between a proximal retracted position in which the at least one expandable ablation member is housed within the sheath to thereby prevent the at least one expandable ablation member from radially expanding from the non-expanded state to the expanded state, and a distal extended position in which the at least one expandable ablation member is exposed from the sheath to thereby permit the at least one expandable ablation member to radially expand from the non-expanded state to the expanded state for contacting tissue.

### EXAMPLE 2

The surgical instrument of Example 1, wherein the at least one expandable ablation member includes at least one electrode.

### EXAMPLE 3

The surgical instrument of Example 2, wherein the at least one expandable ablation member further includes an inflatable balloon, wherein the at least one electrode is positioned on an external surface of the inflatable balloon, wherein an internal cavity of the inflatable balloon is configured to receive an inflation fluid when the ablation catheter is in the distal extended position for radially expanding the at least one expandable ablation member from the non-expanded state to the expanded state.

### EXAMPLE 4

The surgical instrument of Example 2, wherein the at least one electrode comprises a resiliently biased and electrically conductive material.

### EXAMPLE 5

The surgical instrument of Example 4, wherein the at least one electrode is resiliently biased radially outwardly relative to the longitudinal axis such that the at least one electrode is configured to be radially compressed by the sheath when the ablation catheter is in the proximal retracted position, and such that the at least one electrode is configured to resiliently radially expand from the non-expanded state to the expanded state in response to the ablation catheter translating from the proximal retracted position to the distal extended position.

### EXAMPLE 6

The surgical instrument of any one or more of Examples 4 through 5, wherein the at least one electrode has a mesh configuration.

### EXAMPLE 7

The surgical instrument of any one or more of Examples 4 through 6, wherein the ablation catheter includes a shaft defining the longitudinal axis and terminating at a distal tip, wherein the at least one electrode includes a proximal electrode and a distal electrode each disposed on the shaft.

### EXAMPLE 8

The surgical instrument of Example 7, wherein the distal electrode is disposed on the distal tip such that at least a portion of the distal electrode is distal of the distal tip.

### EXAMPLE 9

The surgical instrument of any one or more of Examples 7 through 8, wherein at least one of the proximal electrode or the distal electrode is selectively longitudinally translatable relative to the other of the proximal electrode or the distal electrode along the shaft.

### EXAMPLE 10

The surgical instrument of any one or more of Examples 7 through 9, wherein the distal tip comprises a rigid electrode.

### EXAMPLE 11

The surgical instrument of any one or more of Examples 1 through 10, wherein the ablation catheter includes a navigation sensor, the navigation sensor being operable to generate a signal indicating a position of the navigation sensor within a patient.

### EXAMPLE 12

The surgical instrument of any one or more of Examples 1 through 11, further comprising a camera configured to visualize at least one of an anatomy of the patient's head or the at least one expandable ablation member.

### EXAMPLE 13

The surgical instrument of Example 12, wherein the camera is fixed to a distal end of one of the sheath or the ablation catheter.

### EXAMPLE 14

The surgical instrument of Example 12, wherein the camera is selectively actuatable between a distal extended position and a proximal retracted position relative to the at least one expandable ablation member for selectively visualizing the anatomy of the patient's head and the at least one expandable ablation member, respectively.

### EXAMPLE 15

A surgical system comprising: (a) the surgical instrument of claim 2, wherein the at least one electrode includes a plurality of electrodes; and (b) an RF energy source operatively coupled with the plurality of electrodes; wherein the surgical instrument is operable to energize the plurality of electrodes with RF energy from the RF energy source, wherein the plurality of electrodes are configured to deliver bipolar RF energy to tissue for ablating the tissue.

### EXAMPLE 16

A surgical instrument comprising: (a) a sheath; and (b) an ablation catheter disposed within the sheath, wherein the ablation catheter comprises: (i) a shaft defining a longitudinal axis, and (ii) at least one electrode disposed on the shaft, wherein the at least one electrode is operable to deliver RF energy to tissue for ablating the tissue, wherein the at least one electrode is resiliently biased radially outwardly relative to the longitudinal axis, wherein the ablation catheter is selectively translatable relative to the sheath between a proximal retracted position in which the at least one electrode is housed within the sheath, and a distal extended position in which the at least one electrode is exposed from the sheath, wherein the at least one electrode is configured to resiliently transition from a non-expanded state to an expanded state in response to distal extension of the at least one electrode from the sheath.

### EXAMPLE 17

The surgical instrument of Example 16, wherein the at least one electrode is selectively longitudinally translatable along the shaft.

### EXAMPLE 18

A method of ablating tissue in a head of a patient with an RF ablation instrument, wherein the RF ablation instrument includes a sheath and an ablation catheter disposed within the sheath, wherein the ablation catheter extends along a longitudinal axis and has an expandable ablation member, the method comprising: (a) inserting a distal end of the RF ablation instrument into a head of the patient while the expandable ablation member remains retracted within the sheath; (b) positioning the distal end of the RF ablation instrument at a target region of tissue in the head of the patient; (c) exposing the expandable ablation member relative to the sheath; (d) radially expanding the expandable ablation member from a non-expanded state to an expanded state; (e) contacting the tissue with the exposed, expanded ablation member to place an electrode of the expandable ablation member in electrical contact with the tissue; and (f) energizing the electrode of the expandable ablation member in electrical contact with the tissue with RF energy to thereby ablate the tissue with the RF energy.

### EXAMPLE 19

The method of Example 18, wherein the ablated tissue includes a posterior nasal nerve.

### EXAMPLE 20

The method of any one or more of Examples 18 through 19, wherein radially expanding the expandable ablation member from the non-expanded state to the expanded state includes supplying an inflation fluid to the expandable ablation member after actuating the ablation catheter distally to expose the expandable ablation member from the sheath.

V. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument comprising:
(a) a sheath configured to be inserted into a cavity of a patient's head; and
(b) an ablation catheter disposed within the sheath, wherein the ablation catheter extends along a longitudinal axis and includes at least one expandable ablation member configured to selectively radially expand outwardly from a non-expanded state to an expanded state for selectively ablating tissue within the patient's head,
wherein the ablation catheter is selectively translatable relative to the sheath between a proximal retracted position in which the at least one expandable ablation member is housed within the sheath to thereby prevent the at least one expandable ablation member from radially expanding from the non-expanded state to the expanded state, and a distal extended position in which the at least one expandable ablation member is exposed from the sheath to thereby permit the at least one expandable ablation member to radially expand from the non-expanded state to the expanded state for contacting tissue.

2. The surgical instrument of claim 1, wherein the at least one expandable ablation member includes at least one electrode.

3. The surgical instrument of claim 2, wherein the at least one expandable ablation member further includes an inflatable balloon, wherein the at least one electrode is positioned on an external surface of the inflatable balloon, wherein an internal cavity of the inflatable balloon is configured to receive an inflation fluid when the ablation catheter is in the distal extended position for radially expanding the at least one expandable ablation member from the non-expanded state to the expanded state.

4. The surgical instrument of claim 2, wherein the at least one electrode comprises a resiliently biased and electrically conductive material.

5. The surgical instrument of claim 4, wherein the at least one electrode is resiliently biased radially outwardly relative to the longitudinal axis such that the at least one electrode is configured to be radially compressed by the sheath when the ablation catheter is in the proximal retracted position, and such that the at least one electrode is configured to resiliently radially expand from the non-expanded state to the expanded state in response to the ablation catheter translating from the proximal retracted position to the distal extended position.

6. The surgical instrument of claim 4, wherein the at least one electrode has a mesh configuration.

7. The surgical instrument of claim 4, wherein the ablation catheter includes a shaft defining the longitudinal axis and terminating at a distal tip, wherein the at least one electrode includes a proximal electrode and a distal electrode each disposed on the shaft.

8. The surgical instrument of claim 7, wherein the distal electrode is disposed on the distal tip such that at least a portion of the distal electrode is distal of the distal tip.

9. The surgical instrument of claim 7, wherein at least one of the proximal electrode or the distal electrode is selectively longitudinally translatable relative to the other of the proximal electrode or the distal electrode along the shaft.

10. The surgical instrument of claim 7, wherein the distal tip comprises a rigid electrode.

11. A surgical system comprising:
(a) the surgical instrument of claim 2, wherein the at least one electrode includes a plurality of electrodes; and
(b) an RF energy source operatively coupled with the plurality of electrodes;
wherein the surgical instrument is operable to energize the plurality of electrodes with RF energy from the RF energy source,
wherein the plurality of electrodes are configured to deliver bipolar RF energy to tissue for ablating the tissue.

12. The surgical instrument of claim 1, wherein the ablation catheter includes a navigation sensor, the navigation sensor being operable to generate a signal indicating a position of the navigation sensor within a patient.

13. The surgical instrument of claim 1, further comprising a camera configured to visualize at least one of an anatomy of the patient's head or the at least one expandable ablation member.

14. The surgical instrument of claim 13, wherein the camera is fixed to a distal end of one of the sheath or the ablation catheter.

15. The surgical instrument of claim 13, wherein the camera is selectively actuatable between a distal extended position and a proximal retracted position relative to the at least one expandable ablation member for selectively visualizing the anatomy of the patient's head and the at least one expandable ablation member, respectively.

16. A surgical instrument comprising:
(a) a sheath; and
(b) an ablation catheter disposed within the sheath, wherein the ablation catheter comprises:
(i) a shaft defining a longitudinal axis, and
(ii) at least one electrode disposed on the shaft, wherein the at least one electrode is operable to deliver RF energy to tissue for ablating the tissue, wherein the at least one electrode is resiliently biased radially outwardly relative to the longitudinal axis,
wherein the ablation catheter is selectively translatable relative to the sheath between a proximal retracted position in which the at least one electrode is housed within the sheath, and a distal extended position in which the at least one electrode is exposed from the sheath,
wherein the at least one electrode is configured to resiliently transition from a non-expanded state to an expanded state in response to distal extension of the at least one electrode from the sheath.

17. The surgical instrument of claim 16, wherein the at least one electrode is selectively longitudinally translatable along the shaft.

18. A method of ablating tissue in a head of a patient with an RF ablation instrument, wherein the RF ablation instrument includes a sheath and an ablation catheter disposed within the sheath, wherein the ablation catheter extends along a longitudinal axis and has an expandable ablation member, the method comprising:
(a) inserting a distal end of the RF ablation instrument into a head of the patient while the expandable ablation member remains retracted within the sheath;
(b) positioning the distal end of the RF ablation instrument at a target region of tissue in the head of the patient;
(c) exposing the expandable ablation member relative to the sheath;
(d) radially expanding the expandable ablation member from a non-expanded state to an expanded state;
(e) contacting the tissue with the exposed, expanded ablation member to place an electrode of the expandable ablation member in electrical contact with the tissue; and
(f) energizing the electrode of the expandable ablation member in electrical contact with the tissue with RF energy to thereby ablate the tissue with the RF energy.

19. The method of claim 18, wherein the ablated tissue includes a posterior nasal nerve.

20. The method of claim 18, wherein radially expanding the expandable ablation member from the non-expanded state to the expanded state includes supplying an inflation fluid to the expandable ablation member after actuating the ablation catheter distally to expose the expandable ablation member from the sheath.
